(19) United States
(12) Patent Application Publication
(10) Pub. No.: US 2022/0310239 A1
(43) Pub. Date: Sep. 29, 2022

(54) CONFIGURATION AND SPACE MANAGEMENT OF PATIENT SUPPORT APPARATUS

(71) Applicant: International Business Machines Corporation, Armonk, NY (US)

(72) Inventors: Venkata Vara Prasad Karri, Visakhapatnam (IN); Sarbajit K. Rakshit, Kolkata (IN)

(21) Appl. No.: 17/213,450

(22) Filed: Mar. 26, 2021

(51) Int. Cl. G16H 40/20; G16H 10/60; G16H 50/70; G16H 40/40; G16H 50/20; A61G 7/05

(52) U.S. Cl. CPC G16H 40/20; G16H 10/60; G16H 50/70; G16H 40/40; G16H 50/20; A61G 7/0528; A61G 2203/30; A61G 2203/36; A61G 2203/40; A61G 2203/22; G16Y 10/75

(57) ABSTRACT

A system for autonomous patient support apparatuses is provided. A computing device receives: (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses. The computing device predicts an expected number of additional patients to be admitted to the healthcare facility. The computing device generates a request to modify a configuration of one or more autonomous patient support apparatuses within a patient treatment room based, at least in part, on the patient data, the autonomous patient support apparatus data, and the expected number of additional patients. The computing device instructs the one or more autonomous patient support apparatuses to modify the configuration of the one or more autonomous patient support apparatuses within the patient treatment room.

### BACKGROUND OF THE INVENTION

The present invention relates generally to the field of intelligent facilities and space management systems, and more particularly to the use of artificial intelligence for autonomous/semi-autonomous patient support apparatuses.

Generally, in the field of healthcare, patient support apparatuses (such as hospital beds) are positioned and moved manually by healthcare staff within the confines of a healthcare facility. As technology has evolved, artificial intelligence has been used in various ways to help improve patient healthcare.

### SUMMARY

Embodiments of the present invention provide a method, system, and program product.

A first embodiment encompasses a method. One or more processors receives (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses. The one or more processors predict an expected number of additional patients to be admitted to the healthcare facility within a particular timeframe. The one or more processors generate a request to modify a configuration of one or more autonomous patient support apparatuses within a patient treatment room of the healthcare facility based, at least in part, on the patient data, the autonomous patient support apparatus data, and the expected number of additional patients. The one or more processors instruct the one or more autonomous patient support apparatuses to modify the configuration of the one or more autonomous patient support apparatuses within the patient treatment room based, at least in part, on the generated request.

A second embodiment encompasses a computer program product. The computer program product includes one or more computer-readable storage media and program instructions stored on the one or more computer-readable storage media. The program instructions include program instructions to receive (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses. The program instructions include program instructions to predict an expected number of additional patients to be admitted to the healthcare facility within a particular timeframe. The program instructions include program instructions to generate a request to modify a configuration of one or more autonomous patient support apparatuses within a patient treatment room of the healthcare facility based, at least in part, on the patient data, the autonomous patient support apparatus data, and the expected number of additional patients. The program instructions include program instructions to instruct the one or more autonomous patient support apparatuses to modify the configuration of the one or more autonomous patient support apparatuses within the patient treatment room based, at least in part, on the generated request.

A third embodiment encompasses a computer system. The computer system includes one or more computer processors, one or more computer-readable storage media, and program instructions stored on the computer-readable storage media for execution by at least one of the one or more processors. The program instructions include program instructions to receive (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses. The program instructions include program instructions to predict an expected number of additional patients to be admitted to the healthcare facility within a particular timeframe. The program instructions include program instructions to generate a request to modify a configuration of one or more autonomous patient support apparatuses within a patient treatment room of the healthcare facility based, at least in part, on the patient data, the autonomous patient support apparatus data, and the expected number of additional patients. The program instructions include program instructions to instruct the one or more autonomous patient support apparatuses to modify the configuration of the one or more autonomous patient support apparatuses within the patient treatment room based, at least in part, on the generated request.

### BRIEF DESCRIPTION OF THE SEVERAL VIEWS OF THE DRAWINGS

FIG. 1 is a functional block diagram illustrating a computing environment, in which a system configures a group of client devices, in accordance with an exemplary embodiment of the present invention.

FIG. 2 illustrates operational processes of executing a system generating a request to configure the layout of the client devices, on a computing device within the environment of FIG. 1, in accordance with an exemplary embodiment of the present invention.

FIG. 3 illustrates operational processes of executing a system for manipulating the configuration of a singular client device, on a computing device within the environment of FIG. 1, in accordance with an exemplary embodiment of the present invention.

FIG. 4 depicts a block diagram of a group of client devices being positioned from a static formation, according to at least one embodiment of the present invention.

FIG. 5 depicts a block diagram of an isolated client device, according to at least one embodiment of the present invention.

FIG. 6 depicts a cloud computing environment, according to at least one embodiment of the present invention.

FIG. 7 depicts abstraction model layers, according to at least on embodiment of the present invention.

FIG. 8 depicts a block diagram of components of one or more computing devices within the computing environment depicted in FIG. 1, in accordance with an exemplary embodiment of the present invention.

### DETAILED DESCRIPTION

Detailed embodiments of the present invention are disclosed herein with reference to the accompanying drawings. It is to be understood that the disclosed embodiments are merely illustrative of potential embodiments of the present invention and may take various forms. In addition, each of the examples given in connection with the various embodiments is intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments of the present invention recognize that in a modernized digital environment, technology can be utilized to provide patients with a threshold level of exceptional care. Embodiments of the present invention provide for a more efficient and comfortable experience for patients by utilizing autonomous patient support apparatuses. Furthermore, embodiments of the present invention provide for a system to configure patient support apparatuses to provide a threshold level of efficiency for medical staff and personnel to attend to patients and to maximize the space allocation of patient support apparatuses within a defined area of space (e.g., patient treatment rooms).

Embodiments of the present invention provide a technological improvement over known solutions for autonomous patient support apparatuses within a healthcare facility. Embodiments of the present invention provide servers and systems that improve over conventional systems by providing a more efficient, and computationally efficient, autonomous IoT system that reduces the overall system load. Embodiments of the present invention provide a system that determines the necessary amount of patient support apparatuses for a given area and further determines that unused patient support apparatuses should remain dormant in storage, thereby reducing computational load. Embodiments of the present invention recognize that the incorporation of patient data allows for the system to analyze patient vitals in real time and allow for hospital staff to reduce time spent maneuvering between each patient's physical location, which again, reduces overall system load.

Embodiments of the present invention provide that patient data that is being accessed by administrator program 122 is being utilized in compliance with various regulatory requirements that include, but are not limited to, HIPAA (Health Insurance Portability and Accountability Act) and other federal, state, and local statutes and regulations. Embodiments of the present invention further provide that in some embodiments patient data is randomly anonymized by a pseudo-random number generator to reduce the risk of mishandling and unintentional disclosure to outside parties (e.g., parties unaffiliated with the healthcare facility and are not medical staff or personnel of the healthcare facility), along with other privacy and/or security measure known (or to be known) in the art.

Embodiments recognize that the driving operations of an autonomous patient support apparatus are controlled, at least in part, by an autonomous driver that comprises a computing device and program instructions that are executed by that computing device. Embodiments recognize an ever increasing confidence level in autonomous patient support apparatuses and the ability for autonomous patient support apparatuses to operate safely and reliably. Embodiments recognize that, in some scenarios, human interaction and judgment are still factors that can affect the safety of a given scenario.

In general, autonomous patient support apparatuses operate using sensors to navigate hallways, walkways, and similar trafficways, as well as the immediate surroundings of the autonomous patient support apparatuses. The autonomous patient support apparatuses often determine the positions of other nearby patient support apparatuses or objects relative the autonomous patient support apparatus and further determine whether the autonomous patient support apparatus should decrease acceleration to zero and stop, accelerate, maintain current speed, and/or change pathway (e.g., change positioning in the trafficway). In general, autonomous patient support apparatuses recognize when objects, persons, or other patient support apparatuses are within the surroundings of the autonomous patient support apparatus and the autonomous patient support apparatus can properly navigate the trafficways by changing positioning, negotiating turns, or by performing various moving maneuvers.

The present invention will now be described in detail with reference to the Figures.

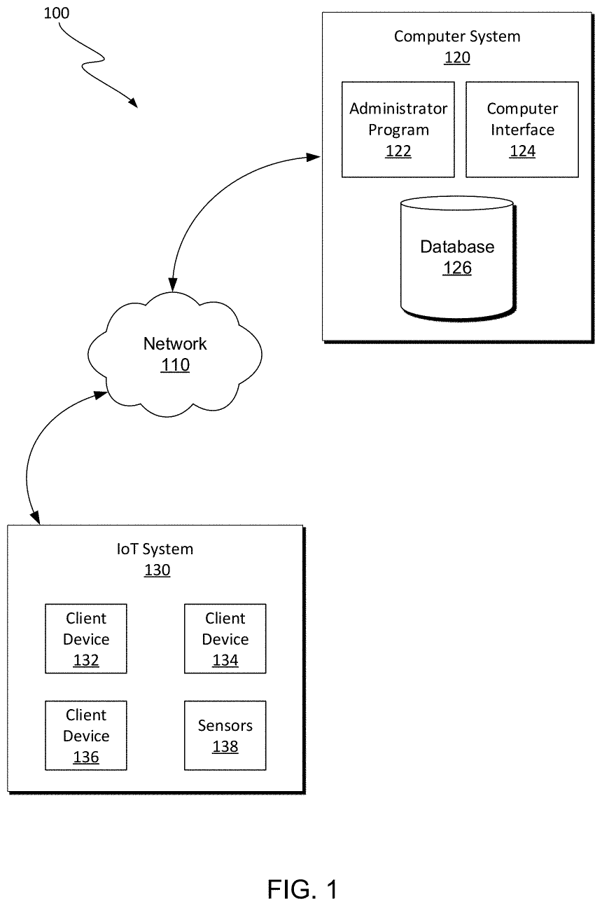

FIG. 1 is a functional block diagram illustrating a computing environment, generally designated 100, in accordance with an embodiment of the present invention. Computing environment 100 includes computer system 120 and IoT system 130. Computer system 120 includes administrator program 122, computer interface 124, and database 126. IoT system 130 130 includes client device 132, client device 134, client device 136, and sensors 138.

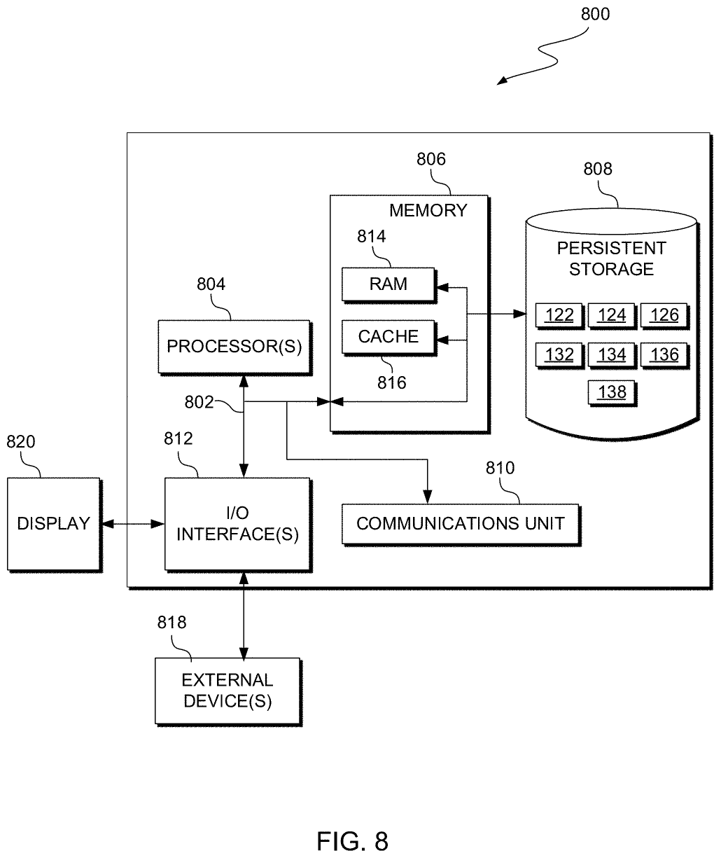

In various embodiments of the present invention, computer system 120 is a computing device that can be a standalone device, a server, a laptop computer, a tablet computer, a netbook computer, a personal computer (PC), a personal digital assistant (PDA), a smartwatch, a desktop computer or any programmable electronic device capable of executing machine readable program instructions and communications with IoT system 130. In another embodiment, computer system 120 represents a computing system utilizing clustered computers and components to act as a single pool of seamless resources. In general, computer system 120 can be any computing device or a combination of devices with access to IoT system 130 and network 110 and is capable of executing administrator program 122, computer interface 124, and database 126. Computer system 120 may include internal and external hardware components as depicted and described in further detail with respect to FIG. 8.

In this exemplary embodiment, administrator program 122 and computer interface 124 are stored on computer system 120. However, in other embodiments, administrator program 122 and computer interface 124 may be stored externally and accessed through a communication network, such as network 110. Network 110 can be, for example, a local area network (LAN), a wide area network (WAN) such as the Internet, or a combination of the two, and may include wired, wireless or any other connection known in the art. In general, network 110 can be any combination of connections and protocols that will support communications between computer system 120 and IoT system 130, in accordance with a desired embodiment of the present invention.

Administrator program 122 is depicted in FIG. 1 for illustrative simplicity. In various embodiments of the present invention, administrator program 122 represents logical operations executing on computer system 120, where computer interface 124 manages the ability to view these logical operations and their results on computer system 120. Computer system 120 can include any number of logical operations that are managed and executed in accordance with administrator program 122. In some embodiments, administrator program 122 represents an administrator that analyzes input and output (I/O) data. Additionally, administrator program 122, when executing hospital bed configurations for patients, operates to monitor the I/O data that was analyzed and generates a modification based on, but not limited to, the analyzation operation. In some embodiments, administrator program 122 determines whether a specific action is likely to take place and generates a modification request and communicates a notification to IoT system 130 indicating that a modification or reconfiguration is required for client device 132, client device 134, and client device 136 (collectively, client devices 132-136) within IoT system 130.

Computer system 120 includes computer interface 124. Computer interface 124 provides an interface between computer system 120 and IoT system 130. In some embodiments, computer interface 124 can be a graphical user interface (GUI), a web user interface (WUI), or an image projector and can display text, documents, web browsers, windows, user options, application interfaces, instructions for operation, images, and holography displays, and includes the information (such as graphic, text, and sound) that a program presents to a user and the control sequences the user employs to control the program. In some embodiments, computer system 120 accesses data communicated from IoT system 130 via a client-based application that runs on computer system 120. For example, computer system 120 includes mobile application software that provides an interface between computer system 120 and IoT system 130.

In various embodiments of the present invention, client device 132, client device 134, and client device 136 (collectively, client devices 132-136) represent autonomous hospital beds and/or autonomous patient support apparatuses capable of executing machine readable program instructions and communications with computer system 120 and within IoT system 130. In various embodiments, computing environment 100 includes additional various client devices (i.e., autonomous patient support apparatuses) not shown. In another embodiment, client devices 132-136 represent a computing system utilizing clustered computers and components to act as a single pool of seamless resources with access to computer system 120 and network 110. IoT system 130 may include internal and external hardware components as depicted and described in further detail with respect to FIG. 8.

Sensors 138 are depicted in FIG. 1 for illustrative simplicity. In various embodiments of the present invention, sensors 138 represent various computing devices executing on client devices 132-136, where sensors 138 communicate data to administrator program 122. In various embodiments, sensors 138 represent computing devices that monitor and determine the surroundings of client devices 132-136 to avoid persons and inanimate objects. Additionally, in various embodiments, sensors 138 determine the locations of various other client devices (e.g., autonomous patient support apparatuses) in order to prevent collisions between the various autonomous patient support apparatuses. In various embodiments, sensors 138 determine whether specific action is likely to take place and generate data that is communicated to administrator program 122, wherein administrator program 122 generates a digital modification and communicates the digital modification to the client devices 132-136. Additionally, various other sensors 138 are present in FIG. 1 (not shown), in which client devices 132-136 utilize one or more sensors 138 to monitor patient data and communicate patient data to administrator 122 to generate a digital modification. In various embodiments of the present invention, sensors 138 include various sensors within one or more client devices, including, but not limited to, (i) one or more proximity sensors which can be used to identify persons, inanimate objects, and various other patient support apparatuses, and (ii) patient sensors that monitor the various vital signs of the one or more patients.

In various embodiments of the present invention, client devices 132-136 include an autonomous patient support apparatus that represents any programmable electronic device capable of executing machine readable program instructions and communicating with computer system 120 and IoT system 130. In some embodiments, client devices 132-136 represent an autonomous computing device capable of moving and positioning the patient support apparatus within the confines of a healthcare facility and monitoring patient vital signs to identify patent data and communicate the patient data to administrator program 122.

In various embodiments of the present invention, an array of cameras and various sensors are positioned within the healthcare facility to monitor the movement of client devices 132-136. In these embodiments, the array of cameras and various sensors capture the progression of the state and/or positions of client devices 132-136 and persons and inanimate objects that might impede the movement and/or positioning of client devices 132-136. This data collected by an array of cameras and various sensors is sent to administrator program 122 for analyzation and comparison to determine whether one or more of client devices 132-136 must move or reposition in accordance with an impediment with a person or inanimate object.

In various embodiments, alternatively, administrator program 122 communicates with database 126 and accesses: (i) data associated with vacant and occupied patient support apparatuses (i.e., client devices 132-136), (ii) patient data regarding one or more admitted patients to the healthcare facility, and (iii) environmental data regarding the healthcare facility and environmental data regarding natural hazards (i.e., geophysical, hydrological, climatological, meteorological, and biological) that impact persons in and around the healthcare facility.

In various embodiments of the present invention, administrator program 122 receives I/O data in the form of (i.e., is included as a part of) patient data and autonomous patient support apparatus data. Administrator program 122 analyzes the data and prepares a digital modification for client devices 132-136 (e.g., patient support apparatuses). In various embodiments, administrator program 122 communicates the data that represents the digital modification with program instructions instructing client devices 132-136 executing within IoT system 130 (i.e., healthcare facility) to move or reposition.

In various embodiments of the present invention, administrator program 122 continuously monitors data received from sensors 138 executing on client devices 132-136 as well as the array of cameras and various other sensors executing within the IoT system 130 (i.e., healthcare facility). In various embodiments, administrator program 122 monitors at various time periods the positioning of client devices 132-136 and patient data received from sensors 138. In various embodiments, administrator program 122 receives a reconfiguration request from a user of computer system 120 where the reconfiguration request instructs administrator program 122 to communicate a set of program instructions to client devices 132-136 instructing client devices 132-136 to position in a specified pattern within a large room within the IoT system 130 (e.g., a large room of a healthcare facility). In various embodiments, administrator program 122 analyzes patient data received from sensors 138 indicating that one of the patients located on a patient support apparatus requires immediate medical attention from a plurality of medical staff and professionals. In this embodiment, administrator program 122 communicates a set of program instructions to client devices 132-136 to reposition to provide additional space and room for the medical staff and professionals to attend to the patient.

In various embodiments, administrator program 122 actively monitors for data received from client devices 132-136 and database 126 (e.g., receives a digital modification). In various embodiments, administrator program 122 receives data from sensors 138, where sensors 138 monitor the environment around the patient support apparatuses and patient vitals to modify, reposition, or rotate one or more patient support apparatuses (e.g., client devices 132-136) within the IoT system 130 (e.g., a healthcare facility). Embodiments of the present invention recognize that sensors 138 monitor the environment of IoT system 130 and identify when obstructions exist within the pathway of the patient support apparatuses during the modification, reconfiguration, or rotation of the patient support apparatuses. In various embodiments, administrator program 122 receives data from sensors 138 and generates a set program instructions instructing client devices 132-136 to alter the pathway of the modification, reconfiguration, or rotation of the patient support apparatuses to avoid the obstructions. In various embodiments, sensors 138 identify patient data regarding the vital signs of the patients occupying the patient support apparatuses (e.g., client devices 132-136) and communicates the patient data to administrator program 122. In various embodiments, administrator program 122 analyzes the patient data received from sensors 138 and determines whether a modification, reconfiguration, and/or rotation of the various occupied patient support apparatuses is required. In some embodiments, administration program 122 determines that a modification, reconfiguration, and/or rotation of the various occupied patient support apparatuses is required and communicates a set of program instructions instructing various patient support apparatuses to modify, reconfigure, or rotate their current positions. Alternatively, administration program 122 determines that a modification, reconfiguration, and/or rotation of the various occupied patient support apparatuses is not required and does not communicate a set of program instructions to the various patient support apparatuses.

In various embodiments of the present invention, FIG. 1 includes one or more sensors (e.g., sensors 138) that monitor one or more patient support apparatuses (e.g., client devices 132-136) to identify data to communicate to administrator program 122. In various embodiments, administrator program 122 analyzes the data received from sensors 138 and determines whether to modify, reconfigure, and/or rotate the patient support apparatuses (i.e., client devices 132-136). In various embodiments, computing environment 100 depicted in FIG. 1 represents a multi-use patient support healthcare facility, wherein one or more patients occupy the various patient support apparatuses (i.e., client devices 132-136).

In some embodiments of the present invention, administrator program 122 receives I/O data in the form of (i.e., is included as a part of): (i) patient data from sensors 138 connected over network 110 and (ii) patient data from database 126, but embodiments of administrator program 122 are not limited thereto. In various embodiments, administrator program 122 analyzes the patient data and identifies: (i) one or more modifications, reconfigurations, and/or rotations to the patient support apparatuses, (ii) whether a specific patient support apparatus should modify, reconfigure, and/or rotate its positioning or trajectory based on, at least, new patient data or an obstruction identified by sensors 138, and/or (iii) whether the modification, reconfiguration, and/or rotation is required for subsequent use.

Embodiments of the present invention provide that administrator program 122 will utilize historical data as training data to determine different configurations for patient support apparatuses and identification of space requirements within the confines of a healthcare facility. In various embodiments, the sensors executing within IoT system 130 monitor patients being treated for a variety of injuries and/or diseases and communicate the data to administrator program 122. Additionally, administrator program 122 identifies patient data that includes one or more of, but is not limited to, treatment plans, mobility information, assigned medical staff and personnel, and/or medical devices. In various embodiments, administrator program 122 utilizes the patient data to establish a configuration of the patient support apparatuses within the confines of a fixed space within the healthcare facility (e.g., a room, auditorium, etc.) with regards to the injuries and/or diseases of the admitted patients. In various embodiments, administrator program 122 communicates data related to the patient to the specific patient support apparatus to allow for autonomous movement. In various embodiments, the administrator program 122 communicates patient data that includes, but is not limited to, treatment plans and identified interaction with medical staff and personnel. In various embodiments, administrator program 122 communicates modification requests to one or more patient support apparatuses (i.e., client devices 132-136) to reconfigure or for a singular patient support apparatus to move to allow more space for medical staff to provide immediate care and attention to the patient. In various embodiments, administrator program 122 operates to communicate program instructions instructing the patient support apparatuses to identify priority of treatment to patients and accordingly allocate space and create passage-ways between the patient support apparatuses to allow medical staff and personnel access to patients supported by the patient support apparatuses.

In various embodiments, if administrator program 122 identifies that vacant patient support apparatuses exceed a threshold value of incoming and/or admitted patients, then administrator program 122 communicates program instructions instructing the vacant patient support apparatuses to remain dormant in storage or to remain in a space distant from the patients being attended to. In various embodiments, administrator program 122 monitors the flow of admitted and discharged patients and removes and adds patient support apparatuses as needed.

In various embodiments, administrator program 122 receives data from sensors 138 related to patient data of each individual patient of the patient support apparatuses. In various embodiments, administrator program 122 identifies the injuries and/or diseases of the various patients from the patient data and generates a modification request to reconfigure the patient support apparatuses so that patients with similar injuries and/or diseases are congregated together to allow medical staff and personnel to treat patients more quickly.

In various embodiments, administrator program 122 receives patient data from the various patient support apparatuses related to admitted patients. In some embodiments, if administrator program 122 determines that one or more patients may be at risk of exposure of a communicable disease of a separate patient, then administrator program 122 generates a modification request and communicates program instructions instructing one or more patient support apparatuses to increase the distances between the one or more patient support apparatuses to reduce exposure to the disease among the various patients. Embodiments of the present invention further provide that administrator program 122 recognizes that some patients require additional accommodation or assistance and accordingly prioritizes the treatment plan and arrangement of their patient support apparatuses near emergency egress and medical staff and/or personnel.

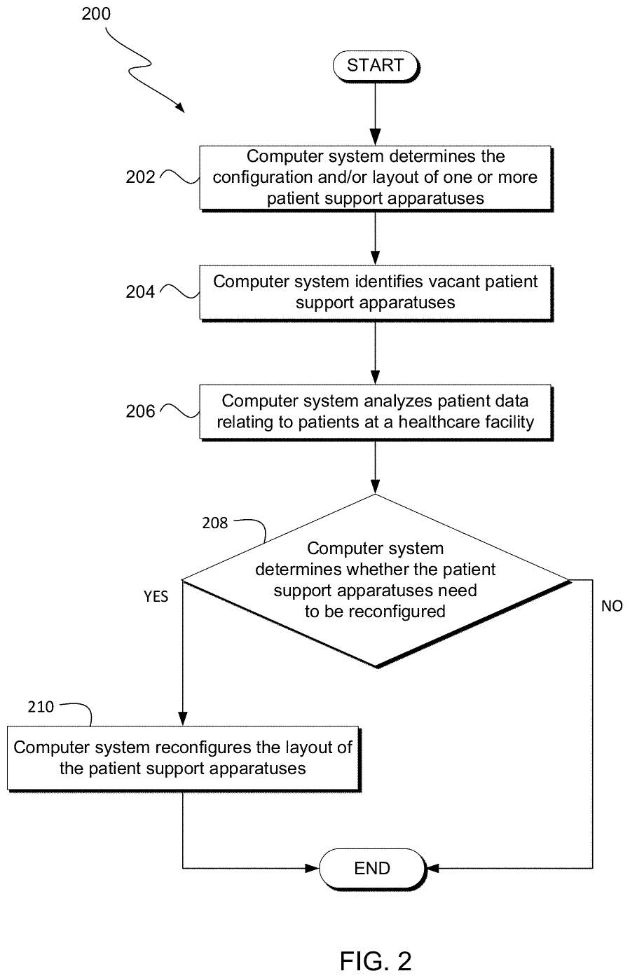

FIG. 2 is a flowchart depicting operations for space and facility utilization of patient support apparatuses within computing environment 100, in accordance with an illustrative embodiment of the present invention. More specifically, FIG. 2, depicts combined overall operations 200, of administrator program 122. In some embodiments, operations 200 represent logical operations of administrator program 122, wherein administrator program 122 represents interactions between logical computing devices communicating with computer system 120 and various other computing devices connected to network 110. It should be appreciated that FIG. 2 provides an illustration of one implementation and does not imply any limitations with regard to the environments in which different embodiments may be implemented. Many modifications to the depicted environment may be made. In one embodiment, the series of operations, in flowchart 200, can be terminated at any operation. In addition to the features previously mentioned, any operations of flowchart 200, can be resumed at any time.

In operation 202, administrator program 122 determines the configuration and/or layout of one or more patient support apparatuses. In various embodiments, administrator program 122 determines the current configuration of the one or more patient support apparatuses, where the current configuration of the one or more patient support apparatuses includes, but is not limited to, currently occupied patient support apparatuses existing within a patient treatment room. In various embodiments, administrator program 122 utilizes patient data to determine an optimal configuration for the patient support apparatuses. In various embodiments, administrator program 122 identifies that there is a threshold value of patient support apparatuses within a healthcare facility, and additionally the patient support apparatuses are to be separated by a threshold level distance from one another. In various embodiments, administrator program 122 determines the dimensions of the patient support apparatuses and further determines how to configure the patient support apparatuses while allocating enough space and distance for the separation between patient support apparatuses. In various embodiments, administrator program 122 identifies that a threshold level of patients will be admitted into a first patient treatment room and will occupy an equal threshold level of patient support apparatuses. Additionally, in various embodiments, administrator program 122 identifies based on, at least, the patient data, that one or more of the threshold level of patients will require additional medical services and attention from medical staff and personnel. In various embodiments, administrator program 122 determines that the one or more patients that require additional medical services will be positioned perpendicular to the plurality of patients along the walls of the patient treatment room, where the one or more patients that require additional medical services can be quickly and easily attended to by the medical staff and personnel.

In various embodiments, administrator program 122 determines the configuration of the patient support apparatuses based on factors that include, but are not limited to, (i) the threshold level of patients, (ii) the dimensions of the patient treatment room, (iii) the severity of the conditions of the patients being admitted, (iv) the natural hazard that occurred, and (v) the medical services that will be required to treat the admitted patients. In various embodiments, administrator program 122 determines the configuration of the patient support apparatuses based on, at least, the configuration factors. In various embodiments, administrator program 122 identifies patient data and the configuration factors to determine a layout that allows for medical staff and personnel the greatest access to all of the patients within the patient treatment room and that will allow the autonomous patient support apparatuses space to maneuver and reconfigure, on an as needed basis. In various embodiments, administrator program 122 identifies the needs of patients based on patient data and configures the patient support apparatuses to provide for a threshold level of care and medical attention from the medical staff and personnel. In various embodiments, administrator program 122 determines an optimal configuration of the one or more patient support apparatuses based on, at least, current patient data. In various embodiments, administrator program 122 utilizes data from the patient data that includes, but not limited to, the current amount of admitted patients and the patient support apparatuses utilized by the current admitted patients, the current configuration of those occupied patient support apparatuses, and the needs of the patients with regards to the amount and time of the medical staff and personnel to attend to the current admitted patients. In various embodiments, administrator program 122 further determines how to integrate additional admitted patients and their associated patients support apparatuses to create an optimal configuration that allows for an equal spacing between patient support apparatuses and for medical staff and personnel to provide a threshold level of care to the admitted patients.

In an alternative embodiment, administrator program 122 determines an optimal configuration based on predicted future data, where administrator program 122 determines an optimal configuration in the event of patients being admitted due to being affected by a natural hazard. In various embodiments, administrator program 122 identifies the predicted value of admitted patients based on, at least, the type of natural hazard and the severity level of the natural hazard. In various embodiments, the severity of the natural hazard represents how impactful the natural hazard was against the general populace and is based on ratings and scales known in the art. In various embodiments, in response to administrator program 122 identifying the predicted value of admitted patients, then administrator program 122 determines an optimal configuration that allows for an equal spacing between patient support apparatuses and for medical staff and personnel to provide a threshold level of care to the admitted patients. In one example embodiment, the natural hazard that occurred was an earthquake of a threshold level on the magnitude scale and administrator program 122 determines that a predicted five-hundred (500) patients will be admitted to the healthcare facility. In this one example embodiment, administrator program 122 determines that there must be a high threshold value of available walk space for the medical staff and personnel to routinely reach all of the admitted patients. In this example embodiment, administrator program 122 first configures a threshold level of patient support apparatuses along the circumference of the patient treatment room, secondly configures a higher threshold level of patient support apparatuses in a first ‘U-shaped’ pattern, and thirdly configures one or more smaller additional ‘U-shaped’ patterns within the first U-shaped pattern. Additionally, in this one example embodiment, administrator program 122 instructs the one or more ‘U-shaped’ patient support apparatuses to be spaced six (6) feet apart to allow for one or more medical staff and personnel to walk in between each ‘U-shaped’ pattern.

In operation 204, administrator program 122 identifies vacant patient support apparatuses. In various embodiments, administrator program 122 identifies the amount of patient support apparatuses occupied by patients within various patient treatment rooms within the healthcare facility. In various embodiments, administrator program 122 determines that a natural hazard (e.g., a geophysical, hydrological, climatological, meteorological, and/or biological event) has occurred and that the healthcare facility expects to admit a threshold level of patients for treatment based on injuries sustained by the natural hazard. In various embodiments, administrator program 122 identifies the threshold level of patient support apparatuses required to meet the threshold level of predicted patients from the natural hazard.

In various embodiments, administrator program 122 determines a predicted threshold level of patient support apparatuses to provide for patients of a natural hazard based on historical training data. In various embodiments, a user provides historical training data to administrator program 122 that identifies that for a first natural hazard that affected a roadway, one-hundred and twenty patients are expected to arrive at the healthcare facility for treatment. In various embodiments, administrator program 122 identifies one-hundred and twenty vacant patient support apparatuses and determines a configuration for the one-hundred and twenty patient support apparatuses distributed throughout various patient treatment rooms. In various embodiments, the one-hundred and twenty potential patients are separated into equal groupings between the various patient treatment rooms based on, at least, the expected severity of their conditions and vitals. In various embodiments, administrator program 122 receives additional historical training data that helps identify a second geophysical hazard where an identified one-thousand patients are expected to arrive at the healthcare facility for treatment. In various embodiments, administrator program 122 identifies one-thousand vacant patient support apparatuses and determines a configuration for the one-thousand patient support apparatuses distributed throughout the healthcare facility. In various embodiments, if administrator program 122 determines that the required number of patient support apparatuses are not available (i.e., not enough vacant patient support apparatuses), then administrator program 122 identifies the threshold level of vacant patient support apparatuses firstly available and additional patient support apparatuses on a rolling basis. Additionally, in various embodiments, administrator program 122 generates an alert and communicates the alert to a user of computer system 120 with program instructions informing the user that there is only a threshold level of patient support apparatuses available, and further instructs the user to navigate the overflow of admitted patients to nearby healthcare facilities. In various embodiments, the distribution of patient support apparatuses is based, at least in part, on: (i) triage units, (ii) severity of condition and vitals of the patient, and/or (iii) assortment of facilities and medical devices required to treat the patients (e.g., surgical suites, etc.).

In various embodiments, administrator program 122 identifies vacant patient support apparatuses based on, at least, (i) patient data stored on database 126 that correlates a patient to a model number of the patient support apparatus and (ii) sensors that communicate patient data to database 126 that identify that a patient is utilizing the patient support apparatus (e.g., weight sensors). In various embodiments, administrator program 122 analyzes the patient data to determines whether a patient support apparatus is vacant. In some embodiments, administrator program 122 determines that patient support apparatus is not vacant based on, at least, the patient data stored on database 126 that correlates an admitted patient to model number of a patient support apparatus. In some embodiments, administrator program 122 analyzes patient support apparatus data on database 126 that indicates that one or more patient support apparatuses are stored in a storage room and administrator program 122 determines that these patient support apparatuses are vacant. In various embodiments, administrator program 122 communicates program instructions to the vacant patient support apparatuses instructing the patient support apparatuses to begin transitioning to patient waiting room and/or the patient treatment room to receive an admitted patient for treatment. In various embodiments, administrator program 122 indicates that the patient support apparatus is vacant and en route to receive an admitted patient. Embodiments of the present invention recognize that at various stages of an admitted patient's treatment cycle the patient may vacate the patient support apparatus for a medical procedure to utilize the restroom or to take a shower or bath. Embodiments of the present invention further recognize that administrator program 122 does not recognize a patient support apparatus as vacant until the admitted patient that was utilizing the patient support apparatus has been formally discharged from the care and service of the healthcare facility, where in response to the admitted patient being discharged administrator program 122 communicates program instructions to the patient support apparatus to return to a sterile storage room for sanitization before being used by another admitted patient.

In operation 206, administrator program 122 analyzes patient data relating to patients at a healthcare facility. In various embodiments, administrator program 122 monitors for patient data received from database 126 and sensors 138. In various embodiments, administrator program 122 continuously monitors for patient data to determine the configuration of the patient support apparatuses. In various embodiments, administrator program 122 receives patient data regarding recently admitted patients to the healthcare facility. In various embodiments, administrator program 122 analyzes the patient data from the recently admitted patients and determines the severity of their condition and vitals, and further determines the treatments that are associated with treating the patients' condition. In various embodiments, administrator program 122 stores the patient data on database 126 for subsequent use by administrator program 122 and/or medical staff and personnel.

In various embodiments, administrator program 122 receives patient data from sensors 138 that execute within IoT system 130. In various embodiments, sensors 138 include, but are not limited to, cameras, video recorders, and proximity sensors that monitor the environment of the patient treatment room and the healthcare facility for a while to identify the various autonomous patient support apparatuses, inanimate objects, and persons moving and positioned within the patient treatment rooms and the healthcare facility as a whole. In various embodiments, sensors 138 execute on the patient support apparatuses and communicate patient data to administrator program 122. In various embodiments, administrator program 122 receives patient data regarding the condition and vitals of the patient occupying the patient support apparatus. In various embodiments, administrator program 122 analyzes the patient data to determine whether the: (i) patient's condition and vitals are stable or (ii) if the patient experiencing a medical emergency.

If administrator program 122 determines that the patient support apparatuses need to be reconfigured, as discussed in FIG. 2 (decision 208, YES branch), then administrator program 122 generates a modification request with program instructions and communicates the modification request with program instructions to the patient support apparatuses. Administrator program 122 determines the modification, reconfiguration, and/or rotation of the one or more patient support apparatuses within the patient treatment room (operation 210). If administrator program 122 determines that patient support apparatuses do not need to be reconfigured, as discussed in FIG. 2 (decision 208, NO branch), then administrator program 122 does not generate a modification request and allows the one or more patient support apparatuses to remain in their current positioning within the patient treatment room.

In operation 210, administrator program 122 reconfigures the layout of the patient support apparatuses. In various embodiments, administrator program 122 continuously monitors patient data received from database 126 and sensors 138 executing within IoT system 130. In various embodiments, administrator program 122 identifies that a first patient from the one or more patients within a patient treatment room is being discharged, then administrator program 122 generates a modification request with program instructions instructing the discharged patient's autonomous patient support apparatus to vacate the patient treatment room and return to the sterile storage room for sanitization. Additionally, administrator program 122 generates a modification request with program instructions instructing the remaining occupied patient support apparatuses to reconfigure and fill in the space left by the patient support apparatus that vacated the patient treatment room. In various embodiments, administrator program 122 identifies that a second patient from the one or more patients within a patient treatment room is experiencing a medical emergency based on, at least, patient data received from sensors 138. In various embodiments, administrator program 122 generates a modification request with program instructions instructing the patient support apparatus providing for the second patient to maneuver and move closer to door of patient treatment room so that medical staff and personnel and quickly and easily assess the patient, additionally, administrator program 122 instructs the remaining patient support apparatuses to reconfigure and provide space for the second patient; patient support apparatus to move through the patient treatment room and to provide space for the medical staff and personnel to enter the room and assess the second patient.

In various embodiments, administrator program 122 identifies that a third patient from the one or more patients within a patient treatment room is either: (i) contagious or (ii) highly susceptible to infection based on, at least patient data, and requires additional distance from the one or more patients within the patient treatment room. In various embodiments, administrator program 122 generates a modification request with program instructions instructing the patient support apparatus of the third patient to maneuver to an isolated location within the patient treatment room, where the patient is given the most space as possible between the third patient and the remaining patients within the patient treatment room. Additionally, administrator program 122 instructs the remaining patient support apparatuses to localize on the opposite side of the patient treatment room from the isolated location of the third patient. Administrator program 122 further instructs the remaining patient support apparatuses to ensure that a threshold level of equal spacing between each patient support apparatus exists and that medical staff and personnel can still easily access each individual patient within the remaining patient support apparatuses.

In various embodiments, administrator program 122 determines that one or more patients and the accompanying patient support apparatuses will be occupying a patient treatment room with previously existing patients and patient support apparatuses. In various embodiments, administrator program 122 generates a modification request with program instructions instructing the previously existing patient support apparatuses to reconfigure and create space for the new patient support apparatuses. In various embodiments, administrator program 122 instructs the previously existing patient support apparatuses to move a threshold level of distance away from the entryway of the patient treatment room, while maintaining the current layout of the previously existing patient support apparatuses. In various embodiments, administrator program 122 instructs the new patient support apparatuses to enter the patient treatment room and occupy the locations where the previously existing patient support apparatuses were prior to the modification request. Embodiments of the present invention recognize that any configuration of patient support apparatuses exist and that the modification requests are generated based on, at least, the necessity for providing space or reallocating the patient support apparatuses based on patient data.

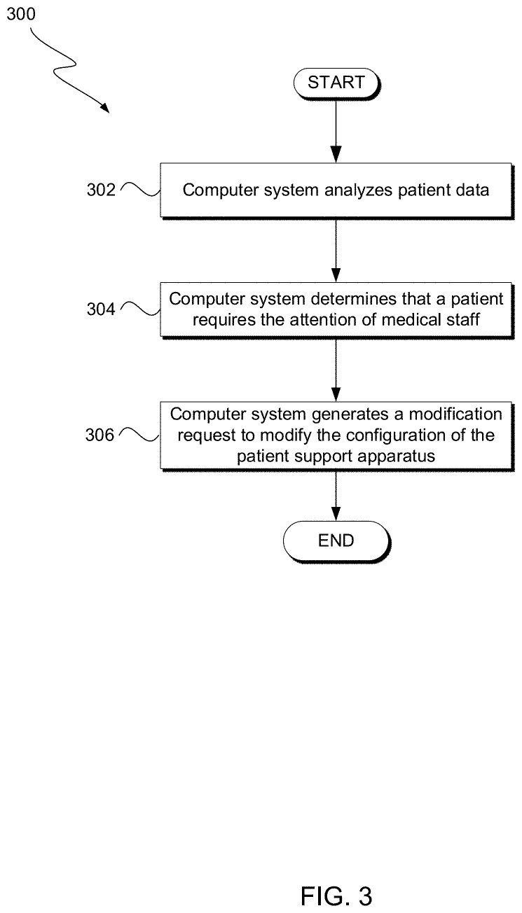

FIG. 3 depicts flowchart, 300, depicting operations of administrator program 122 in computing environment 100, in accordance with an illustrative embodiment of the present invention. More specifically, FIG. 3, depicts combined overall operations, 300, of administrator program 122 executing on computer system 120. In some embodiments, operations 300 represents logical operations of administrator program 122, wherein interactions between administrator program 122, client devices 132-136, and sensors 138 represent logical units executing on computer system 120. Further, operations 300 can include a portion or all of combined overall operations of 200. In various embodiments, the series of operations 300, can be performed at the conclusion of operations 200. In some embodiments, operations 300 can be performed simultaneously with operations 200. It should be appreciated that FIG. 3 provides an illustration of one implementation and does not imply any limitations with regard to the environments in which different embodiments may be implemented. Many modifications to the depicted environment may be made. In one embodiment, the series of operations, of flowchart 300, can be performed simultaneously. Additionally, the series of operations, in flowchart 300 can be terminated at any operation. In addition to the features previously mentioned, any operations of flowchart 300 can be resumed at any time.

In operation 302, administrator program 122 analyzes patient data. In various embodiments, administrator program 122 receives patient data from database 126 and sensors 138 executing within IoT system 130. In various embodiments, administrator program 122 analyzes patient data to determine whether a patient is: (i) experiencing a medical emergency, (ii) the patient is being discharged or is no longer in need of the patient support apparatus, or (iii) if the patient needs to be transported to a separate location within the healthcare facility (i.e., operating theatre, MRI, X-ray, etc.). In various embodiments, administrator program 122 analyzes the patient data for each individual patient within the various patient treatment room and identifies patients that are stable and do not require immediate medical attention or to be transported.

In operation 304, administrator program 122 determines that a patient requires the attention of medical staff. In various embodiments, administrator program 122 determines that a first patient requires medical attention based on, at least, patient data received from database 126 and sensors 138 executing within IoT system 130. In various embodiments, administrator program 122 determines that the first patient is experiencing a medical emergency that requires medical staff to perform emergency operations on the patient within the patient treatment room. In various embodiments, administrator program 122 communicates an alert to the user(s) of computer system 120 and alerts medical staff and personnel to the patient's location utilizing at least, sirens or emergency tones, lights, and/or guided messages directing the medical staff and personnel to the patient's location. Additionally, administrator program 122 identifies whether the patient will need to be transported for further medical procedures based on, at least, the patient data and generates an alert to the user(s) (e.g., if the patient will require surgery, then an alert will be generated to prepare the operating theatre and instruct the necessary medical staff and personnel to the operating theatre). In various embodiments, administrator program 122 generates a privacy request with program instructions that allow for the curtain to be automatically drawn closed to provide privacy for the patient during the medical emergency. Additionally, if the first patient's patient support apparatus is not located near a curtain then administrator program 122 generates a modification request with program instructions instructing the patient support apparatus to reposition behind a curtain within the patient treatment room (operation 306, as discussed in further detail below). In various embodiments, if a location with a curtain is occupied then administrator program 122 communicates a modification request with program instructions instructing the one or more patient support apparatuses to reconfigure and reposition to allow for the patient support apparatus support the first patient to reposition behind the curtain for privacy.

In operation 306, administrator program 122 generates a modification request to modify the configuration of the patient support apparatus. In various embodiments, if administrator program 122 identifies that the various patient support apparatuses need to reconfigure, then administrator program 122 generates a modification request with program instructions instructing one or more patient support apparatuses to reconfigure or reposition within the patient treatment room. In various embodiments, administrator program 122 identified that the first patient requires privacy behind a curtain, if the patient support apparatus is not within the location of a curtain, then administrator program 122 generates a modification request with program instructions instructing the first patient's patient support apparatus to reposition to a location within a curtain. In some embodiments, if there is no vacant location with a curtain, then administrator program 122 generates a modification request with program instructions instructing the nearest occupied patient support apparatus to reconfigure and allow for the first patient's patient support apparatus to occupy the location with a curtain for privacy. In various embodiments, if administrator program 122 identifies that a second patient needs to be isolated within the patient treatment room to allow medical staff and personnel ample room to attend to the second patient, then administrator program 122 generates a modification request with program instructions. In various embodiments, the modification request with program instructions instructs the second patient's patient support apparatus to reposition to an isolated position within the patient treatment room and instruct the various other patient support apparatuses to localize to the opposite side of the patient treatment room while maintaining a threshold level of equal spacing between the individual various other patient support apparatuses.

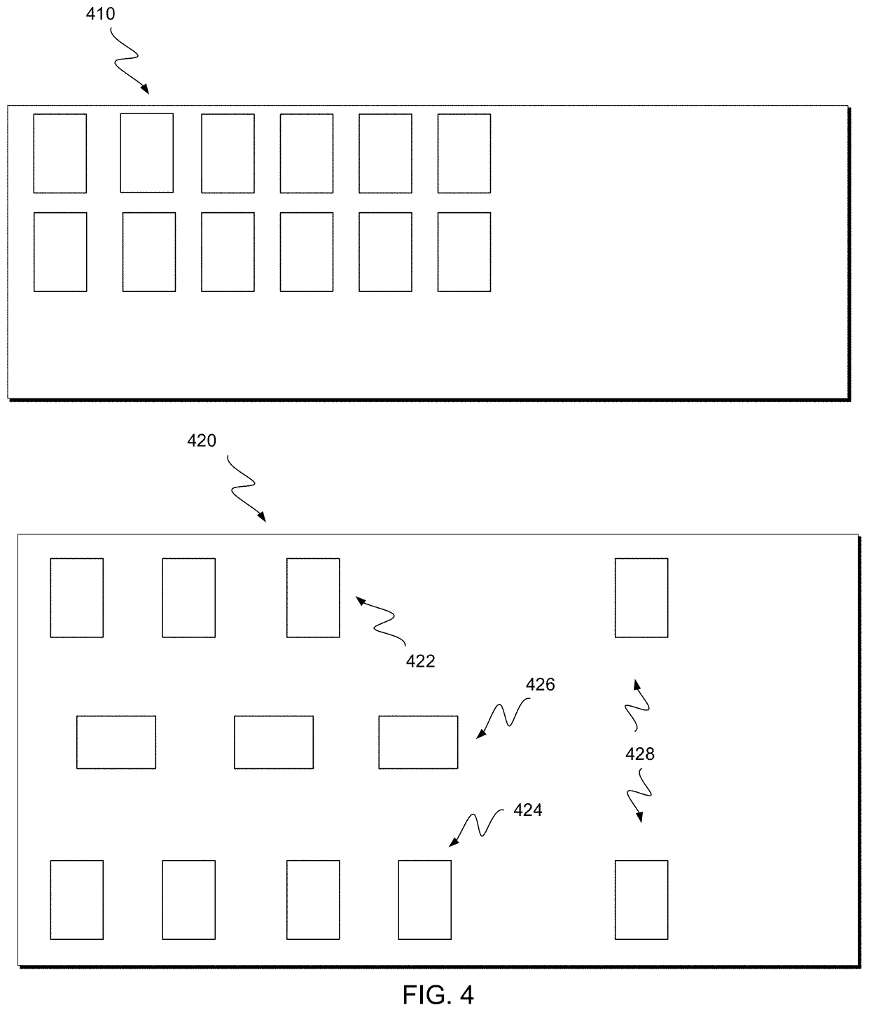

FIG. 4 depicts block diagram 410 and block diagram 420. In various embodiments, block diagram 410 represents a plurality of patient support apparatuses (i.e., client devices 132-136) where the plurality of patient support apparatuses are dormant within storage of the healthcare facility. In various embodiments, the plurality of patient support apparatuses are contained within an area that is sterile and allows for the patient support apparatuses to be sanitized in-between uses by patients admitted to the healthcare facility. In various embodiments, block diagram 420 represents an occupied patient treatment room, where the various patient support apparatuses have been configured throughout the patient treatment room. Embodiments of the present invention provide that the depiction in block diagram 420 is for illustrative simplicity, and that any configuration of any number of patient support apparatuses can exist within the confines of the healthcare facility. In various embodiments, patient support apparatus groups 422 and 424 are located six (6) feet apart from the other patient support apparatuses and are located near integrated medical wall systems. In various embodiments, patient support apparatuses 426 are centrally located within the patient treatment room in a perpendicular positioning to patient support apparatus groups 422 and 424 to allow for medical staff and personnel greater access to the patients of patient support apparatus group 426. In various embodiments, patient support apparatus group 428 is located at a greater distance from patient support apparatus groups 422, 424, and 426 because the patients of patient support apparatus group 428 are in critical condition and are highly susceptible to injury and need to be separately treated and require directed medical attention.

In various embodiments, administrator program 122 monitors IoT system 130 and determines when a patient support apparatus becomes vacant. In various embodiments, administrator program 122 generates a modification request to remove one or more vacant patient support apparatuses from the patient treatment room that houses the patient support apparatuses (i.e., client devices 132-136). In various embodiments, administrator program 122 communicates the modification request with program instructions instructing the one or more patient support apparatuses to vacate the patient treatment room and return to the sterile storage room for sanitization. Additionally, administrator program 122 communicates the modification request with program instructions instructing the remaining occupied patient support apparatuses in the patient treatment room to allocate space for the one or more vacant patient support apparatuses to exit the patient treatment room; and reconfigure the spacing between the remaining occupied patient support apparatuses to allow for equal and safe distancing between the remaining occupied patient support apparatuses and to allow for additional patient support apparatuses to enter and position within the patient treatment room, as needed.

Embodiments of the present invention provide for autonomous patient support apparatuses to be controlled throughout IoT system 130. Additionally, the patient support apparatuses will include: (i) a set of wheels that is capable of rotating in a three-hundred and sixty (36) degrees along the y-axis of an x-y-z plane or (ii) a set of roller sphere wheels that are capable of rotating any rotation along an x-y-z plane. Additionally, the wheel-well of the set of wheels will include motion-detection and proximity sensors to detect the distance of the patient support apparatus to various objects and persons that exist within the healthcare facility. In various embodiments, the set of wheels will have an internal locking mechanism that allows for the wheels to remain in a locked and fixed-state so that the patient support apparatus is not inadvertently moved or adjusted from its current position. In various embodiments, the patient support apparatus will include a plurality of sensors (i.e., sensors 138) that monitor the condition of the patient (i.e., patient data) and communicate the patient data to administrator program 122, wherein the sensors may include, but are not limited to: (i) a heartbeat monitor, (ii) an oxygen saturation monitor, (iii) a blood-glucose monitor, (iv) an infusion pump, (v) a body temperature monitor, and (vi) a respirator. In various embodiments, the IoT system that the patient support apparatuses reside within will monitor the relative positioning of each patient support apparatus and the direction and speed that each patient support apparatus is traveling along. Additionally, the IoT system will monitor the movement and positioning of medical staff, personnel, and patients and visitors within the healthcare facility. In various embodiments, administrator program 122 will determine the allocation of space for the patient support apparatuses based on historical training data from database 126 including, but not limited to, (i) movement patterns of medical staff and personnel, (ii) positioning of medical equipment, and (iii) historical movement patterns of patients (e.g., travel to operating theatres, medical examinations, etc.).

In various embodiments, administrator program 122 will identify historical training data that identifies a threshold level of space between patient support apparatuses that will allow: (i) medical staff and personnel to move in-between patient support apparatuses, (ii) for medical staff and personnel to visit and monitor the condition of the patient from time to time, and (iii) for medical staff and personnel to attend to a patient who may be experiencing a medical emergency.

In various embodiments, administrator program 122 will identify historical training data regarding patient data and configurations of patient support apparatuses that will promote a positive system to allow medical staff and personnel to attend to a threshold level of patients admitted due in part to a natural hazard. In various embodiments, administrator program 122 will identify historical training patient data regarding one or more patient's condition (e.g., using an emergency severity index, which includes five (5) levels: one (1) being most urgent and (5) being least urgent). In various embodiments, administrator program 122 identifies the range of the patient's conditions from least urgent to most urgent (i.e., using historical training patient data) and determines a configuration of patient support apparatuses that: (i) provides medical staff the ability to attend to patients who require a threshold level of urgent care and (ii) provides for patients who do not require a threshold level of urgent care but are positioned within a threshold level proximate distance to patients that require urgent care, wherein medical staff and personnel and efficiently attend to those patients who do not require urgent care.

In various embodiments, administrator program 122 operates to analyze historical patient data and configuration of patient support apparatuses to provide a threshold level of efficiency for medical staff and personnel to attend to patients and to maximize the space allocation of patient support apparatuses within a defined area of space (i.e., patient treatment room). In various embodiments, administrator program 122 will analyze the patient data received from database 126 regarding admitted patients and patients being admitted after the occurrence of a natural hazard and will determine the required amount of patient support apparatuses and which patients should be allocated a patient support apparatus if the threshold level of patients admitted exceeds the availability of vacant patient support apparatuses. In various embodiments, administrator program 122 identifies when patients vacate the use of the patient support apparatuses and communicates program instructions instructing the patient support apparatuses to return to a sterile storage room for sanitization. In various embodiments, administrator program 122 will continually monitor patient data received from database 126 and sensors 138 executing on IoT system 130, and administrator program 122 determine when to modify, reconfigure, and rotate patient support apparatuses within the patient treatment room. Additionally, in various embodiments, administrator program 122 generates program instructions instructing the autonomous patient support apparatuses to monitor the environment around each individual patient support apparatus to ensure that the threshold level of space (i.e., six feet of space in any direction between the patient support apparatuses) is provided between each patient support apparatus and the various object and persons that may enter and move around the patient treatment room. In various embodiments, administrator program 122 will identify patients experiencing similar or like injuries and/or illnesses and will communicate modification requests with program instructions instructing the patient support apparatuses supporting these patients to be configured together in clusters and/or in the identical patient treatment rooms.

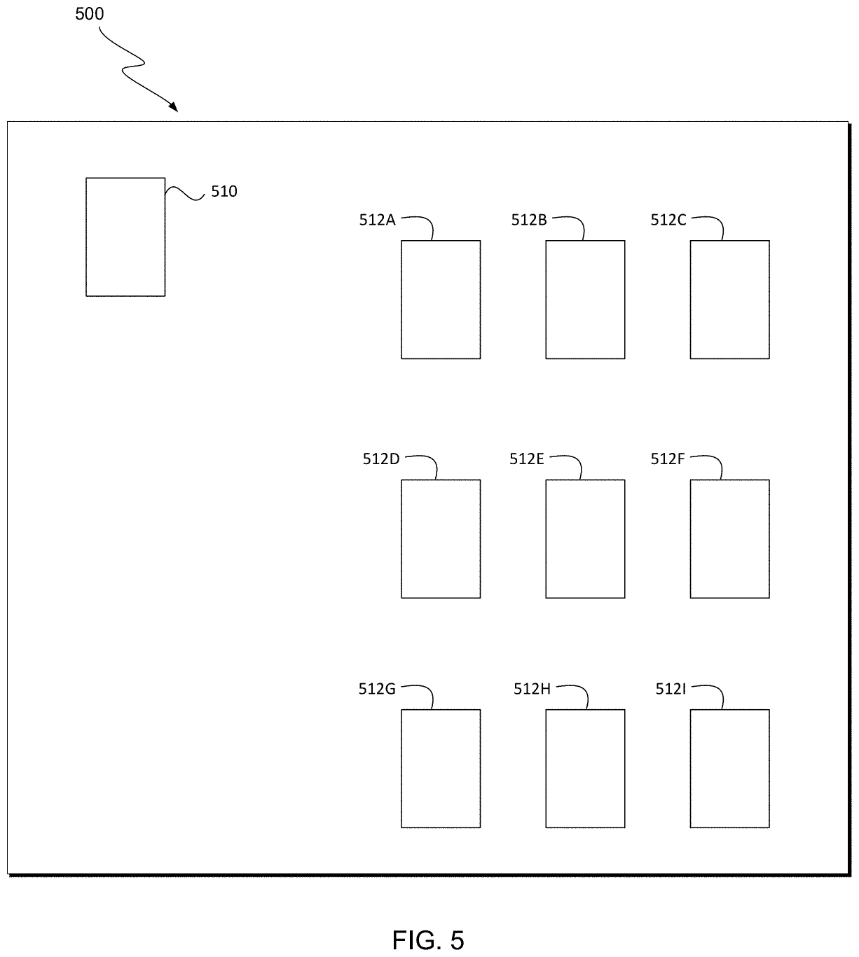

FIG. 5 is a block diagram depicting patient support apparatuses 510 and 512A-512I. In various embodiments, administrator program 122 receives patient data from sensors 138 executing on the patient support apparatuses 510 and 512A-512I. In various embodiments, administrator program 122 initially configures patient support apparatuses 510 and 512A-512I in a pattern (not shown) that provides for equal spacing in between the patient support apparatuses to allow for medical staff and personnel access to attend to the patients, as needed. In various embodiments, administrator program 122 receives updated patient data regarding the patient occupying patient support apparatus 510. In various embodiments, administrator program 122 determines that the patient occupying patient support apparatus 510 is experiencing a medical emergency and requires isolation and/or greater distance from the various other patients. In various embodiments, administrator program 122 generates a modification request with program instructions instructing patient support apparatus 510 to reconfigure and provide greater breadth from patient support apparatuses 512A-512I, additionally, administrator program 122 communicates the modification request with program instructions to patient support apparatuses 512A-512I to reconfigure to allow space for medical staff and personnel to attend to the patient occupying patient support apparatus 510. In various embodiments, patient support apparatus 510 is reconfigured at a threshold level distance from patient support apparatuses 512A-512I.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

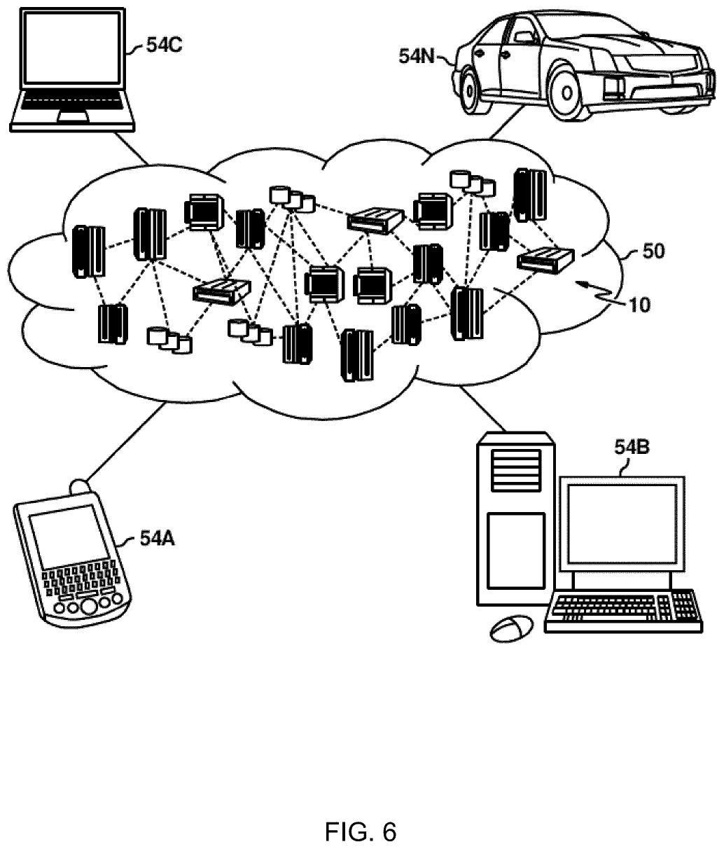

Referring now to FIG. 6, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

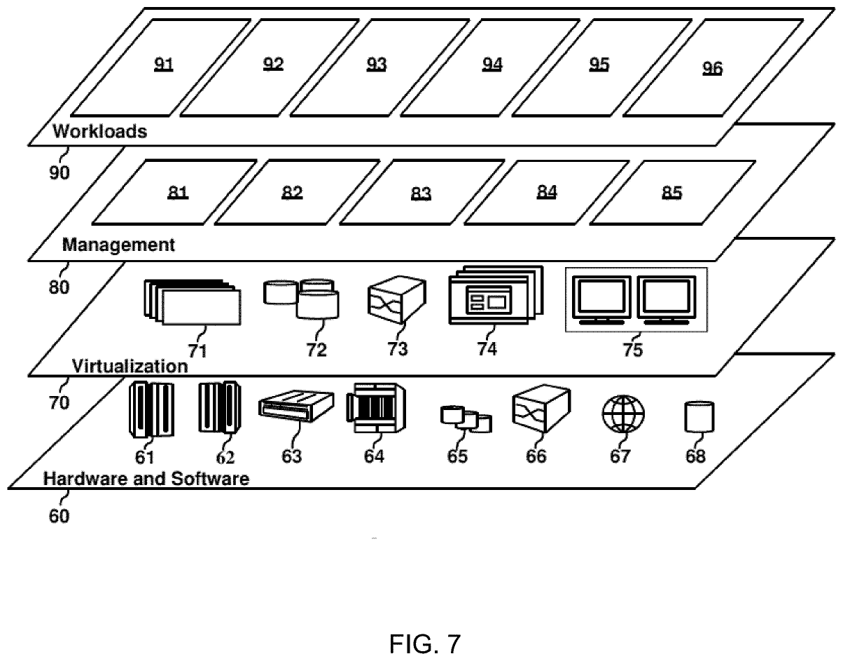

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and providing soothing output 96.

FIG. 8 depicts a block diagram, 800, of components of computer system 120 and client IoT system 130, in accordance with an illustrative embodiment of the present invention. It should be appreciated that FIG. 8 provides only an illustration of one implementation and does not imply any limitations with regard to the environments in which different embodiments may be implemented. Many modifications to the depicted environment may be made.

Computer system 120 IoT system 130 includes communications fabric 802, which provides communications between computer processor(s) 804, memory 806, persistent storage 808, communications unit 810, and input/output (I/O) interface(s) 812. Communications fabric 802 can be implemented with any architecture designed for passing data and/or control information between processors (such as microprocessors, communications and network processors, etc.), system memory, peripheral devices, and any other hardware components within a system. For example, communications fabric 802 can be implemented with one or more buses.

Memory 806 and persistent storage 808 are computer-readable storage media. In this embodiment, memory 806 includes random access memory (RAM) 814 and cache memory 816. In general, memory 806 can include any suitable volatile or non-volatile computer-readable storage media.

Administrator program 122, computer interface 124, database 126, client device 132, client device 134, client device 136, and sensors 138 are stored in persistent storage 808 for execution and/or access by one or more of the respective computer processors 804 via one or more memories of memory 806. In this embodiment, persistent storage 808 includes a magnetic hard disk drive. Alternatively, or in addition to a magnetic hard disk drive, persistent storage 808 can include a solid state hard drive, a semiconductor storage device, read-only memory (ROM), erasable programmable read-only memory (EPROM), flash memory, or any other computer-readable storage media that is capable of storing program instructions or digital information.

The media used by persistent storage 808 may also be removable. For example, a removable hard drive may be used for persistent storage 808. Other examples include optical and magnetic disks, thumb drives, and smart cards that are inserted into a drive for transfer onto another computer-readable storage medium that is also part of persistent storage 808.

Communications unit 810, in these examples, provides for communications with other data processing systems or devices, including resources of network 110. In these examples, communications unit 810 includes one or more network interface cards. Communications unit 810 may provide communications through the use of either or both physical and wireless communications links. Administrator program 122, computer interface 124, database 126, client device 132, client device 134, client device 136, and sensors 138 may be downloaded to persistent storage 808 through communications unit 810.

I/O interface(s) 812 allows for input and output of data with other devices that may be connected to computer system 120 and IoT system 130. For example, I/O interface 812 may provide a connection to external devices 818 such as a keyboard, keypad, a touch screen, and/or some other suitable input device. External devices 818 can also include portable computer-readable storage media such as, for example, thumb drives, portable optical or magnetic disks, and memory cards. Software and data used to practice embodiments of the present invention, e.g., administrator program 122, computer interface 124, database 126, client device 132, client device 134, client device 136, and sensors 138, can be stored on such portable computer-readable storage media and can be loaded onto persistent storage 808 via I/O interface(s) 812. I/O interface(s) 812 also connect to a display 820.

Display 820 provides a mechanism to display data to a user and may be, for example, a computer monitor, or a television screen.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

It is to be noted that the term(s) such as, for example, “Smalltalk” and the like may be subject to trademark rights in various jurisdictions throughout the world and are used here only in reference to the products or services properly denominated by the marks to the extent that such trademark rights may exist.

What is claimed is:

1. A computer-implemented method, the method comprising:
receiving, by one or more processors, (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses;
predicting, by one or more processors, an expected number of additional patients to be admitted to the healthcare facility within a particular timeframe;
generating, by one or more processors, a request to modify a configuration of one or more autonomous patient support apparatuses within a patient treatment room of the healthcare facility based, at least in part, on the patient data, the autonomous patient support apparatus data, and the expected number of additional patients; and
instructing, by one or more processors, the one or more autonomous patient support apparatuses to modify the configuration of the one or more autonomous patient support apparatuses within the patient treatment room based, at least in part, on the generated request.

2. The computer-implemented method of claim 1, wherein generating the request further comprises:
identifying, by one or more processors, one or more vacant autonomous patient support apparatuses based, at least in part, on the occupancy status of the autonomous patient support apparatus data;
identifying, by one or more processors, a configuration of the one or more vacant autonomous patient support apparatuses based, at least in part, on the configuration information of the autonomous patient support apparatus data;
assigning, by one or more processors, one or more of the expected number of additional patients to the one or more vacant autonomous patient support apparatuses; and
determining, by one or more processors, a modification to the configuration of the one or more vacant autonomous patient support apparatuses based, at least in part, on the assigned one or more of the expected number of additional patients.

3. The computer-implemented method of claim 1, wherein generating the request further comprises:
determining, by one or more processors, a medical emergency is occurring with at least one patient based, at least in part, on the patient data; and
generating, by one or more processors, the request to modify the configuration of the one or more autonomous patient support apparatuses based, at least in part, on determining that the medical emergency is occurring.

4. The computer-implemented method of claim 3, the method further comprising:
determining, by one or more processors, to distance an autonomous patient support apparatus occupied by the patient experiencing the medical emergency away from one or more other occupied autonomous patient support apparatuses,
wherein the generated request localizes the one or more other occupied autonomous patient support apparatuses to a first section of the patient treatment room and further localizes the patient support apparatus occupied by the patient experiencing the medical emergency to a second section of the patient treatment room.

5. The computer-implemented method of claim 1, wherein the generating of the request further comprises:
determining, by one or more processors, that a first patient occupying a first autonomous patient support apparatus is scheduled for a medical procedure based, at least in part, on the patient data and the autonomous patient support apparatus data; and
generating, by one or more processors, the request to modify the configuration of the one or more autonomous patient support apparatuses based, at least in part, on determining that the first patient occupying the first autonomous patient support apparatus is scheduled for the medical procedure.

6. The computer-implemented method of claim 1, wherein the generated request reconfigures the one or more autonomous patient support apparatuses within the patient treatment room to provide a threshold level of space between the one or more patient support apparatuses.

7. The computer-implemented method of claim 1, the method further comprising:
determining, by one or more processors, that a patient has vacated an autonomous patient support apparatus within the patient treatment room;
determining, by one or more processors, to send the vacated autonomous patient support apparatus to a storage room; and
generating, by one or more processors, a second request to modify the configuration of the one or more autonomous patient support apparatuses within the patient treatment room, wherein the second request: (i) instructs the vacated autonomous patient support apparatus to move to the storage room, and (ii) instructs one or more remaining autonomous patient support apparatuses within the patient treatment room to reconfigure based on the movement of the vacated autonomous patient support.

8. A computer program product, the computer program product comprising:
one or more computer-readable storage media and program instructions stored on the one or more computer-readable storage media, the stored program instructions comprising:
program instructions to receive: (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses;
program instructions to predict an expected number of additional patients to be admitted to the healthcare facility within a particular timeframe;
program instructions to generate a request to modify a configuration of one or more autonomous patient support apparatuses within a patient treatment room of the healthcare facility based, at least in part, on the patient data, the autonomous patient support apparatus data, and the expected number of additional patients; and
program instructions to instruct the one or more autonomous patient support apparatuses to modify the configuration of the one or more autonomous patient support apparatuses within the patient treatment room based, at least in part, on the generated request.

9. The computer program product of claim 8, the stored program instructions further comprising:
program instructions to identify one or more vacant autonomous patient support apparatuses based, at least in part, on the occupancy status of the autonomous patient support apparatus data;
program instructions to identify a configuration of the one or more vacant autonomous patient support apparatuses based, at least in part, on the configuration information of the autonomous patient support apparatus data;
program instructions to assign one or more of the expected number of additional patients to the one or more vacant autonomous patient support apparatuses; and
program instructions to determine a modification to the configuration of the one or more vacant autonomous patient support apparatuses based, at least in part, on the assigned one or more of the expected number of additional patients.

10. The computer program product of claim 8, wherein the program instructions to generate the request further comprise:
program instructions to determine a medical emergency is occurring with at least one patient based, at least in part, on the patient data; and
program instructions to generate the request to modify the configuration of the one or more autonomous patient support apparatuses based, at least in part, on determining that the medical emergency is occurring.

11. The computer program product of claim 10, the stored program instructions further comprising:
program instructions to determine to distance an autonomous patient support apparatus occupied by the patient experiencing the medical emergency away from one or more other occupied autonomous patient support apparatuses,
wherein the generated request localizes the one or more other occupied autonomous patient support apparatuses to a first section of the patient treatment room and further localizes the patient support apparatus occupied by the patient experiencing the medical emergency to a second section of the patient treatment room.

12. The computer program product of claim 8, wherein the program instructions to generate the request further comprise:
program instructions to determine that a first patient occupying a first autonomous patient support apparatus is scheduled for a medical procedure based, at least in part, on the patient data and the autonomous patient support apparatus data; and
program instructions to generate the request to modify the configuration of the one or more autonomous patient support apparatuses based, at least in part, on determining that the first patient occupying the first autonomous patient support apparatus is scheduled for the medical procedure.

13. The computer program product of claim 8, wherein the generated request reconfigures the one or more autonomous patient support apparatuses within the patient treatment room to provide a threshold level of space between the one or more patient support apparatuses.

14. The computer program product of claim 8, the stored program instructions further comprising:
program instructions to determine that a patient has vacated an autonomous patient support apparatus within the patient treatment room;
program instructions to determine to send the vacated autonomous patient support apparatus to a storage room; and
program instructions to generate a second request to modify the configuration of the one or more autonomous patient support apparatuses within the patient treatment room, wherein the second request: (i) instructs the vacated autonomous patient support apparatus to move to the storage room, and (ii) instructs one or more remaining autonomous patient support apparatuses within the patient treatment room to reconfigure based on the movement of the vacated autonomous patient support.

15. A computer system, the computer system comprising:
one or more processors;
one or more computer readable storage medium; and
program instructions stored on the computer readable storage medium for execution by at least one of the one or more processors, the stored program instructions comprising:
program instructions to receive: (i) patient data relating to patients at a healthcare facility, and (ii) autonomous patient support apparatus data relating to autonomous patient support apparatuses at the healthcare facility, the autonomous patient support apparatus data including configuration information and occupancy status for the autonomous patient support apparatuses;
program instructions to predict an expected number of additional patients to be admitted to the healthcare facility within a particular timeframe;
program instructions to generate a request to modify a configuration of one or more autonomous patient support apparatuses within a patient treatment room of the healthcare facility based, at least in part, on the patient data, the autonomous patient support apparatus data, and the expected number of additional patients; and
program instructions to instruct the one or more autonomous patient support apparatuses to modify the configuration of the one or more autonomous patient support apparatuses within the patient treatment room based, at least in part, on the generated request.

16. The computer system of claim 15, the stored program instructions further comprising:
program instructions to identify one or more vacant autonomous patient support apparatuses based, at least in part, on the occupancy status of the autonomous patient support apparatus data;
program instructions to identify a configuration of the one or more vacant autonomous patient support apparatuses based, at least in part, on the configuration information of the autonomous patient support apparatus data;
program instructions to assign one or more of the expected number of additional patients to the one or more vacant autonomous patient support apparatuses; and
program instructions to determine a modification to the configuration of the one or more vacant autonomous patient support apparatuses based, at least in part, on the assigned one or more of the expected number of additional patients.

17. The computer system of claim 15, wherein the program instructions to generate the request further comprise:
program instructions to determine a medical emergency is occurring with at least one patient based, at least in part, on the patient data; and
program instructions to generate the request to modify the configuration of the one or more autonomous patient support apparatuses based, at least in part, on determining that the medical emergency is occurring.

18. The computer system of claim 17, the stored program instructions further comprising:
program instructions to determine to distance an autonomous patient support apparatus occupied by the patient experiencing the medical emergency away from one or more other occupied autonomous patient support apparatuses,
wherein the generated request localizes the one or more other occupied autonomous patient support apparatuses to a first section of the patient treatment room and further localizes the patient support apparatus occupied by the patient experiencing the medical emergency to a second section of the patient treatment room.

19. The computer system of claim 15, wherein the program instructions to generate the request further comprise:
program instructions to determine that a first patient occupying a first autonomous patient support apparatus is scheduled for a medical procedure based, at least in part, on the patient data and the autonomous patient support apparatus data; and
program instructions to generate the request to modify the configuration of the one or more autonomous patient support apparatuses based, at least in part, on determining that the first patient occupying the first autonomous patient support apparatus is scheduled for the medical procedure.

20. The computer system of claim 15, the stored program instructions further comprising:
program instructions to determine that a patient has vacated an autonomous patient support apparatus within the patient treatment room;
program instructions to determine to send the vacated autonomous patient support apparatus to a storage room; and
program instructions to generate a second request to modify the configuration of the one or more autonomous patient support apparatuses within the patient treatment room, wherein the second request: (i) instructs the vacated autonomous patient support apparatus to move to the storage room, and (ii) instructs one or more remaining autonomous patient support apparatuses within the patient treatment room to reconfigure based on the movement of the vacated autonomous patient support.
